(19) United States
(12) Patent Application Publication
(10) Pub. No.: US 2025/0262442 A1
(43) Pub. Date: Aug. 21, 2025

(54) BLE PROGRAMMER INSTRUCTION CLASSES

(71) Applicant: Cardiac Pacemakers, Inc., St. Paul, MN (US)

(72) Inventors: Kevin G. Wika, Blaine, MN (US); James Frank Hiebert, Delano, MN (US); Jonathan H. Kelly, Woodbury, MN (US); Moira B. Sweeney, St. Paul, MN (US); Robert J. Parkinson, Woodbury, MN (US)

(21) Appl. No.: 19/055,334

(22) Filed: Feb. 17, 2025

Related U.S. Application Data
(60) Provisional application No. 63/555,625, filed on Feb. 20, 2024.

(51) Int. Cl. A61N 1/372; A61N 1/39; G16H 40/67; H04W 4/80

(52) U.S. Cl. CPC A61N 1/37223; A61N 1/37252; A61N 1/3968; G16H 40/67; H04W 4/80

(57) ABSTRACT

Systems and methods are disclosed to an ambulatory medical device including an inductive link, a radio frequency (RF) link, and a control circuit. The inductive link includes a coil antenna configured to receive a near field communication signal using mutual inductance. The RF link includes an RF antenna configured to receive an RF communication signal during a communication session. The control circuit is configured to decode an instruction received in the RF communication signal, wherein a class of the instruction requires the communication session to be started using the inductive link when performing the instruction; perform the instruction when the communication session is started using the inductive link and reject the instruction when the communication session is started using the RF link.

### CLAIM OF PRIORITY

This application claims the benefit of U.S. Provisional Application No. 63/555,625, filed on Feb. 20, 2024, which is hereby incorporated by reference in its entirety.

### TECHNICAL FIELD

This document relates generally to medical devices and more particularly to systems, methods, and devices for wireless communication between medical devices.

### BACKGROUND

Ambulatory medical devices (AMDs), including implantable, subcutaneous, wearable, or one or more other medical devices, etc., can monitor, detect, or treat, various conditions including, among other things, heart failure (HF), fibrillation, and myocardial infarction. AMDs can be used to treat patients using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Patient treatment can be adjusted by changing parameters related to detection of the patient's condition and the therapy provided by the device. Patient status can be monitored by uploading diagnostic information from the device.

### SUMMARY

Systems and methods are disclosed for ambulatory medical devices (AMDs) with both a near-field telemetry link and a far-field telemetry link.
In a first Example (Example 1), an AMD includes an inductive link including a coil antenna configured to receive a near field communication signal using mutual inductance, a radio frequency (RF) link including an RF antenna configured to receive an RF communication signal during a communication session, and a control circuit configured to decode an instruction received in the RF communication signal. The instruction may be of a class of instruction that requires the communication session to be started using the inductive link when performing the instruction. The control circuit performs an instruction of that class when the communication session is started using the inductive link, and rejects performing the instruction of that class when the communication session is started using the RF link.
In Example 2, the subject matter of Example 1 optionally includes control circuit configured to decode another instruction received in another RF communication signal during another communication session, wherein a class of the other instruction does not require the communication session to be started using the inductive link when performing the instruction, and perform the other instruction when the communication session is started using either the RF link or the inductive link.
In Example 3, the subject matter of one or both of Examples 1 and 2 optionally includes a control circuit configured to authenticate a role of a second device sending the RF communication signal, and override the rejecting the instruction according to the role of the second device and perform the instruction when the communication session is started using the RF link.
In Example 4, the subject matter of one or any combination of Examples 1-3 optionally includes a control circuit configured to determine that the instruction received in the RF communication signal is a programming instruction to program one or more operating parameters of the AMD, perform the programming instruction when the communication session is started using the inductive link, and reject the programming instruction when the communication session is started using the RF link.
In Example 5, the subject matter of one or any combination of Examples 1-4 optionally includes a therapy circuit operatively coupled to the control circuit and configured to provide an electrical therapy, and a control circuit configured to determine that the instruction received in the RF communication signal is a programming instruction to program a value of a therapy parameter of the electrical therapy provided by the AMD, perform the programming instruction when the communication session is started using the inductive link, and reject the programming instruction when the communication session is started using the RF link.
In Example 6, the subject matter of one or any combination of Examples 1-5 optionally includes a control circuit configured to determine that the instruction received in the RF communication signal is an upload instruction to upload an amount of information stored in the AMD greater than a predetermined threshold amount of information, perform the upload instruction when the communication session is started using the inductive link, and reject the upload instruction when the communication session is started using the RF link.
In Example 7, the subject matter of one or any combination of Examples 1-6 optionally includes a control circuit configured to determine that the instruction received in the RF communication signal is an instruction that when performed results in a communication session lasting longer than a predetermined threshold amount of time, perform the instruction when the communication session is started using the inductive link, and reject the instruction when the communication session is started using the RF link.
In Example 8, the subject matter of one or any combination of Examples 1-7 optionally includes a control circuit configured to determine that the instruction received in the RF communication signal is an update instruction to update firmware in the AMD, perform the update instruction when the communication session is started using the inductive link, and reject the update instruction when the communication session is started using the RF link.
In Example 9, the subject matter of one or any combination of Examples 1-8 optionally includes a control circuit that includes a timeout timer and is configured to begin performing the instruction when the communication session is started using the inductive link, detect interruption of an RF communication signal on the RF link, and resume performing the instruction when another near field communication signal is received on the inductive link before the timeout timer expires.
In Example 10, the subject matter of one or any combination of Examples 1-9 optionally includes a control circuit configured to authenticate a role of a second device sending the RF communication signal, and override the rejecting the instruction when the role of the second device is authenticated, and the instruction programs a parameter included in a profile of parameters enabled for programming according to the role of the second device.
In Example 11, the subject matter of one or any combination of Examples 1-10 optionally includes a control circuit configured to authenticate a role of a second device sending the RF communication signal, and perform the instruction when the communication session is started using the RF link and the role of the second device is authenticated.
In Example 12, the subject matter of one or any combination of Examples 1-11 optionally includes a control circuit configured to perform the instruction when the communication session is started using the RF link and the instruction enables a predetermined profile of operating parameters of the AMD.
Example 13 includes subject matter (such as a method of operating an AMD to communicate with an external device) or can optionally be combined with one or any combination of Examples 1-10 to include such subject matter, comprising receiving an instruction via an RF communication link of the AMD as part of a communication session with a second device, determining that a class of the received instruction requires the communication session to be started using an inductive link of the AMD when performing the instruction, performing the instruction when the communication session is started using the inductive link, and rejecting the instruction when the communication session is started using the RF link.

1In Example 14, the subject matter of Example 13 optionally includes receiving a different instruction via the RF link from the second device, wherein a class of the different instruction does not require the communication session to be started using the inductive link when performing the instruction; and performing, by the AMD, the different instruction when the communication session is started using either the RF link or the inductive link.
In Example 15, the subject matter of one or both of Examples 13 and 14 optionally includes authenticating a role of the second device, and overriding the rejecting the instruction according to the role of the second device and performing the instruction when the communication session is started using the RF link.
In Example 16, the subject matter of one or any combination of Examples 13-15 optionally includes determining that the instruction received via the RF link is a programming instruction to program one or more operating parameters of the AMD, and requiring the communication session to be started using the inductive link to perform the programming instruction.
In Example 17, the subject matter of one or any combination of Examples 13-16 optionally includes determining that the instruction received via the RF link is a programming instruction to program a value of a therapy parameter of an electrical therapy provided by the AMD and requiring the communication session to be started using the inductive link to perform the programming instruction.
In Example 18, the subject matter of one or any combination of Examples 11-15 optionally includes determining that the instruction received via the RF link is an upload instruction to upload an amount of information stored in the AMD greater than a predetermined threshold amount of information and requiring the communication session to be started using the inductive link to perform the upload instruction.
Example 19 includes subject matter (such as an external medical device) or can optionally be combined with one or any combination of Examples 1-18 to include such subject matter, comprising an inductive link including a coil antenna configured to transmit a near field communication signal to AMD using mutual inductance, an RF link including an RF antenna configured to transmit an RF communication signal with the AMD, and a control circuit operatively coupled to the inductive link and the RF link. The control circuit is configured to begin a communication session with the AMD using the inductive link when sending a first class of instruction to the AMD, begin the communication session with the AMD using the RF link when sending a second class of instruction to the AMD, and send authentication information to the AMD during the communication session for authentication of the external medical device.
In Example 20, the subject matter of claim 19 optionally includes a control circuit configured to begin the communication session with the AMD using the near field communication signal when sending a programming instruction to program one or more operating parameters of the AMD.

These non-limiting Examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an example of a patient management system.

FIG. 2 is an example of an ambulatory medical device (AMD).

FIG. 3 is a block diagram of an example of portions of an AMD.

FIG. 4 is a block diagram of an example of portions of an external device included in a patient management system.

FIG. 5 is a flow diagram of an example of a method of operating a communication link of an AMD.

### DETAILED DESCRIPTION

Ambulatory medical devices (AMDs), including implantable, subcutaneous, insertable, wearable, or one or more other medical devices, etc. AMDs can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient. The present inventors have recognized, among other things, devices, systems, and methods to provide a customizable wireless telemetry link to transfer information between an AMD and a separate device.

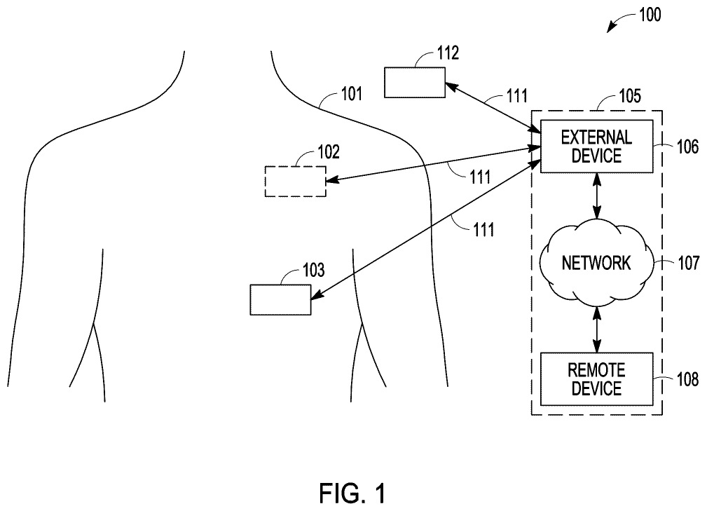

FIG. 1 illustrates an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more medical devices, an external system 105, and a communication link 111 providing for communication between the one or more ambulatory medical devices and the external system 105. The one or more medical devices can include an ambulatory medical device (AMD), such as an implantable medical device (IMD) 102, insertable cardiac monitor (ICM), a wearable medical device 103, or one or more other implantable, leadless, subcutaneous, external, wearable, or medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 of FIG. 1 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 can include a monitor implanted, for example, subcutaneously in the chest of patient 101, the IMD 102 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemakers can communicate between themselves, or one or more other implanted or external devices.

The IMD 102 can include an assessment circuit configured to detect or determine specific physiologic information of the patient 101, or to determine one or more conditions or provide information or an alert to a user, such as the patient 101 (e.g., a patient), a clinician, or one or more other caregivers or processes, such as described herein. The implantable medical device 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the implantable medical device 102 or one or more of the other ambulatory medical devices, etc. In some examples, therapy can include CRT for rectifying dyssynchrony and improving cardiac function in heart failure patients. In other examples, the implantable medical device 102 can include a drug delivery system, such as a drug infusion pump to deliver drugs to the patient for managing arrhythmias or complications from arrhythmias, hypertension, hypotension, or one or more other physiologic conditions. In other examples, the implantable medical device 102 can include one or more electrodes configured to stimulate the nervous system of the patient or to provide stimulation to the muscles of the patient airway, etc.

The wearable medical device 103 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 105 can include a dedicated hardware/software system, such as a medical device programmer, a remote server-based patient management system, or alternatively a system defined predominantly by software running on a standard personal computer. The external system 105 can manage the patient 101 through the implantable medical device 102 or one or more other ambulatory medical devices connected to the external system 105 via a communication link 111. In other examples, the IMD 102 can be connected to the wearable medical device 103, or the wearable medical device 103 can be connected to the external system 105, via the communication link 111. This can include, for example, programming the IMD 102 to perform one or more of acquiring physiologic data, performing at least one self-diagnostic test (such as for a device operational status), analyzing the physiologic data, or optionally delivering or adjusting a therapy for the patient 101. Additionally, the external system 105 can send information to, or receive information from, the IMD 102 or the wearable medical device 103 via the communication link 111. Examples of the information can include real-time or stored physiologic data from the patient 101, diagnostic data, such as detection of patient hydration status, hospitalizations, responses to therapies delivered to the patient 101, or device operational status of the implantable medical device 102 or the wearable medical device 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth, Bluetooth Low Energy (BLE), or IEEE 602.11 wireless fidelity “Wi-Fi” interfacing standards. Other configurations and combinations of patient data source interfacing are possible. For example, a medical device can include more than one type of communication link.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 include a medical device programmer that can monitor patient information collected by AMDs or program the AMD making changes that can impact the patient or a monitoring-only device, such as a patient monitor, etc., that only monitors patient information collected by AMDs but cannot make changes to the device that can impact the patient. The remote device 108 can be configured to evaluate collected patient or patient information and provide alert notifications, among other possible functions. In an example, the remote device 108 can include a centralized server acting as a central hub for collected data storage and analysis from a number of different sources.

Combinations of information from the multiple sources can be used to make determinations and update individual patient status or to adjust one or more alerts or determinations for one or more other patients. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or both of the external device 106 and the remote device 108 can output the detected medical events to a system user, such as the patient or a clinician, or to a process including, for example, an instance of a computer program executable in a microprocessor or other processor. In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The system includes a therapy device 112 that can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 112. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

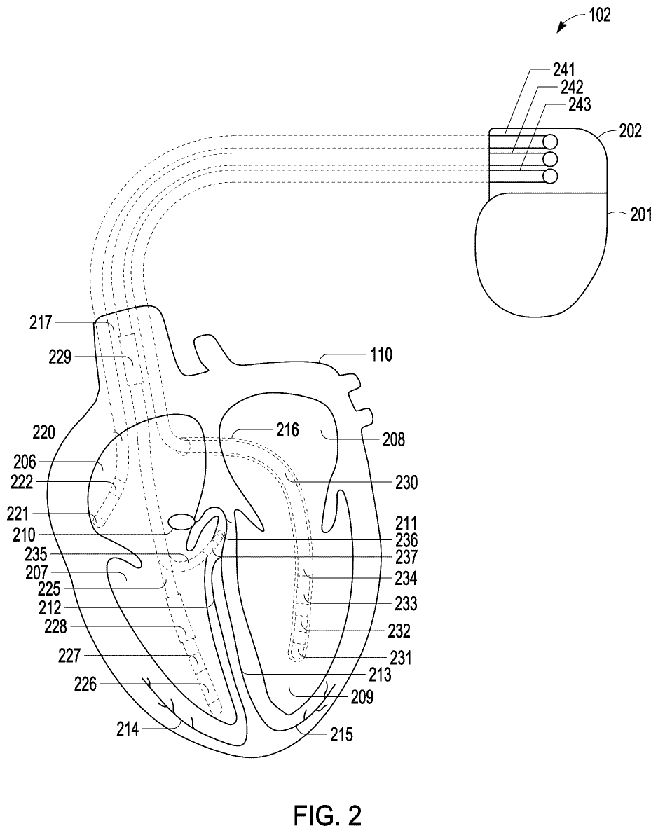

FIG. 2 illustrates an example of an ambulatory medical device that is an IMD 102. The IMD 102 is electrically coupled to a heart 110, such as through one or more leads coupled to the IMD 102 through one or more lead ports, such as first, second, or third lead ports 241, 242, 243 in a header 202 of the IMD 102. In an example, the IMD 102 can include an antenna, such as in the header 202, configured to enable communication with an external system and one or more electronic circuits in a hermetically sealed housing (CAN) 201. The IMD 102 illustrates an example medical device (or a medical device system) as described herein.

The IMD 102 may be an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 110, such as in one or more of the atria or ventricles. Separate from, or in addition to, the one or more electrodes or other sensors of the leads, the IMD 102 can include one or more electrodes or other sensors (e.g., a pressure sensor, an accelerometer, a gyroscope, a microphone, etc.) powered by a power source in the IMD 102. The one or more electrodes or other sensors of the leads, the IMD 102, or a combination thereof, can be configured detect physiologic information from, or provide one or more therapies or stimulation to, the patient.

The IMD 102 can include one or more electronic circuits configured to sense one or more physiologic signals, such as an electrogram or a signal representing mechanical function of the heart 110. In certain examples, the CAN 201 may function as an electrode such as for sensing or pulse delivery. For example, an electrode from one or more of the leads may be used together with the CAN 201 such as for unipolar sensing of an electrogram or for delivering one or more pacing pulses. A defibrillation electrode (e.g., the first defibrillation coil electrode 228, the second defibrillation coil electrode 229, etc.) may be used together with the CAN 201 to deliver one or more cardioversion/defibrillation pulses.

In an example, the IMD 102 can sense impedance such as between electrodes located on one or more of the leads or the CAN 201. The IMD 102 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the IMD 102 can be configured to inject current between an electrode on one or more of the first, second, third, or fourth leads 220, 225, 230, 235 and the CAN 201, and to sense the resultant voltage between the same or different electrodes and the CAN 201.

The example lead configurations in FIG. 2 include first, second, and third leads 220, 225, 230 in traditional lead placements in the right atrium (RA) 206, right ventricle (RV) 207, and in a coronary vein 216 (e.g., the coronary sinus) over the left atrium (LA) 208 and left ventricle (LV) 209, respectively, and a fourth lead 235 positioned in the RV 207 near the His bundle 211, between the AV node 210 and the right and left bundle branches 212, 213 and Purkinje fibers 214, 215. Each lead can be configured to position one or more electrodes or other sensors at various locations in or near the heart 110 to detect physiologic information or provide one or more therapies or stimulation.

The first lead 220, positioned in the RA 206, includes a first tip electrode 221 located at or near the distal end of the first lead 220 and a first ring electrode 222 located near the first tip electrode 221. The second lead 225 (dashed), positioned in the RV 207, includes a second tip electrode 226 located at or near the distal end of the second lead 225 and a second ring electrode 227 located near the second tip electrode 226. The third lead 230, positioned in the coronary vein 216 over the LV 209, includes a third tip electrode 231 located at or near the distal end of the third lead 230, a third ring electrode 232 located near the third tip electrode 231, and two additional electrodes 233, 234. The fourth lead 235, positioned in the RV 207 near the His bundle 211, includes a fourth tip electrode 236 located at or near the distal end of the fourth lead 235 and a fourth ring electrode 237 located near the fourth tip electrode 236. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 225 includes a first defibrillation coil electrode 228 located near the distal end of the second lead 225 in the RV 207 and a second defibrillation coil electrode 229 located a distance from the distal end of the second lead 225, such as for placement in or near the superior vena cava (SVC) 217.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 211.

The IMD 102 is battery-powered and can communicate to an external device (e.g., external device 106 in FIG. 1) using a communication link (e.g., the communication link 111 in FIG. 1). The communication link provides for data transmission from IMD 102 to the external device. This can include, for example, transmitting real-time physiological data acquired by IMD 102, extracting physiological data acquired by and stored in the IMD 102, extracting therapy history data stored in the IMD 102, or extracting data indicating an operational status of the IMD 102 (e.g., battery status and lead impedance). Communication link also provides for data transmission from the external device to the IMD 102. This can include, for example, programming the IMD 102 to acquire physiological data, programming the IMD 102 to perform at least one self-diagnostic test (such as for a device operational status), or programming the IMD to deliver one or more therapies. The communication link can include both a near-field communication link and a far-field communication link.

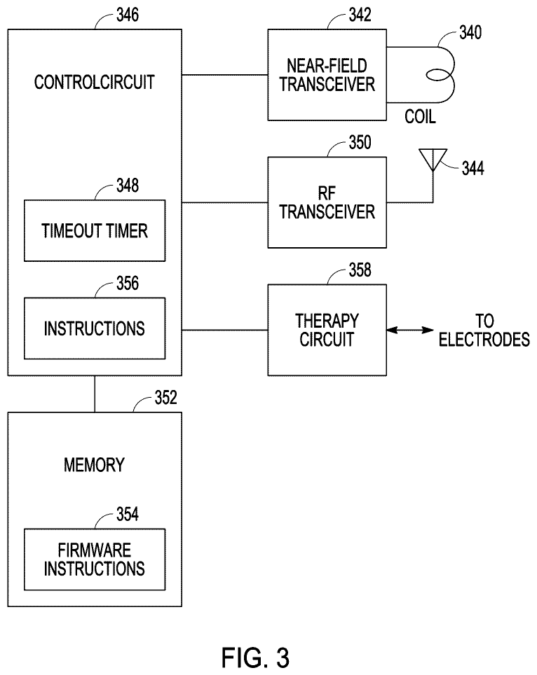

FIG. 3 is a block diagram of portions of an example of an AMD (e.g., any of the AMDs described herein). The communications link includes an inductive (near-field) link and an RF (far-field) link. The inductive link includes a coil antenna 340 and a near-field transceiver 342. The inductive link is configured to receive and send near-field communication signals by mutual inductance with the coil antenna of a second device (e.g., external device 106 in FIG. 1). The RF link includes an RF antenna 344 and an RF transceiver 350. In the IMD 102 of FIG. 2, the coil antenna 340 can be included in the header 202 or around the periphery of the CAN 201. The RF antenna 344 can be included in the header 202 of the IMD 102.

The external device that communicates with the AMD of FIG. 3 (e.g., external device 106 in FIG. 1) may include one or both of an inductive link and an RF link. For the inductive link, energy may be transferred from the coil antenna of the external device to the coil antenna 340 of the AMD via mutual inductance linking the two coil antennas. Data is transferred by sending data bits over the inductive link. For the RF link, the external device may communicate data to the AMD using a Bluetooth Low Energy (BLE) protocol or a Wi-Fi protocol.

The AMD includes a control circuit 346. The control circuit 346 may be implemented using an application-specific integrated circuit (ASIC) constructed to perform one or more functions or a general-purpose circuit programmed to perform the functions. A general-purpose circuit can include, among other things, a microprocessor or a portion thereof, a microcontroller or portions thereof, and a programmable logic circuit or a portion thereof. The control circuit 346 controls the mode of the inductive link and the RF link (e.g., between transmitting and receiving). The control circuit 346 also decodes instructions received via the inductive link and instructions received via the RF link.

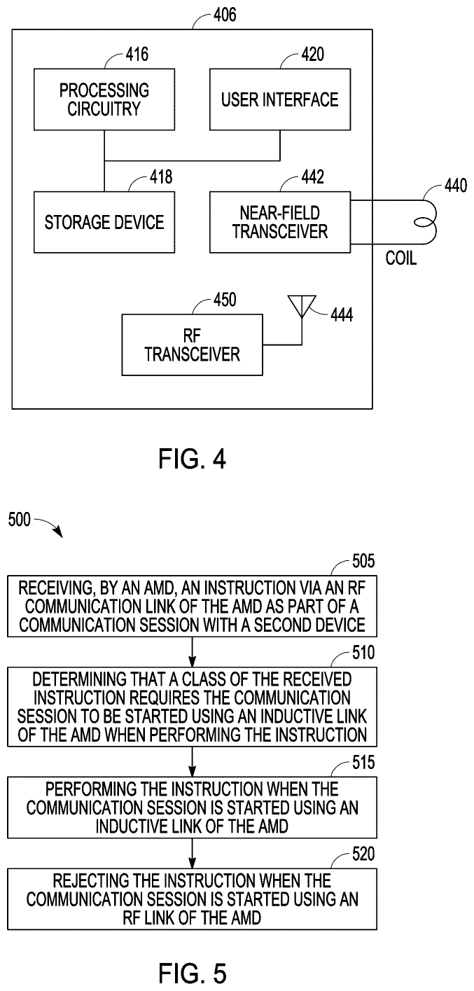

FIG. 4 is a block diagram of an example of portions of an external device 406 included in an external system (e.g., external system 105 in FIG. 1). The external device 406 includes a storage device 418 and processing circuitry 416. In some examples, the external device 406 includes a user interface 420. Processing circuitry 416 may be implemented using an ASIC constructed to perform one or more functions or a general-purpose circuit programmed to perform the functions. The storage device 418 may be a memory integral to the processing circuitry 416, or a separate memory device. The external device 406 includes an inductive link and an RF link to communicate information with another device. Inductive link includes a near-field transceiver 442 operatively coupled to inductive coil antenna 440 and may communicate information wirelessly with the inductive link of FIG. 3 using near-field inductive wireless signals. RF link includes RF antenna 444 and RF transceiver 450. Although disclosed herein as having both the inductive link and the RF link, in certain examples, different types of external devices, such as a monitoring-only device, a patient monitor, etc., can include only the RF link (and not the inductive link or components thereof). In contrast, a medical device programmer can include both the inductive link and the RF link, such as described herein with respect to the external device 406.

The external device 406 can be used to program pacing therapy parameters and other information in an AMD, and upload information stored in the AMD. For the inductive link, the coil antennas of the external device 406 and the AMD are placed proximate to one another so that energy generated in the coil antenna 440 of the external device 406 creates energy in the coil antenna 440 of the AMD. Presence of energy at a predetermined time corresponds to a “one” bit and absence of energy at a predetermined time corresponds to a “zero” bit. In this way, the inductive link is a serial communication link. The processing circuitry 415 may include a near-field protocol driver that defines the content and order of the bytes of data transmitted to the AMD and the content and order of the bytes of data received from the AMD. For the RF link, the external device may communicate data to the AMD using a BLE protocol as described previously herein. The processing circuitry 415 may include a far-field protocol driver that provides data to the AMD and receives data from the AMD according to the BLE protocol or a Wi-Fi protocol.

A communication session between the external device 406 and AMD may begin using either the inductive link or the RF link. A communication session is typically started by the external device 406 sending an interrogation message and the AMD sending a response message. In some examples, a communication is started using the inductive link and then is switched to the RF link. The external device 406 may transmit interrogation messages using the inductive link until a response message is received from the AMD on the inductive link. The external device 406 may then optionally enable the far-field RF link to further communicate with the AMD. This is useful during a procedure implanting an AMD to first identify the AMD with near-field telemetry and then communicate using far-field telemetry after the identification. This allows the near-field telemetry to wake-up the AMD, but the far-field telemetry allows communication while the AMD is in a sterile field without requiring a coil antenna of a telemetry “wand” to enter the sterile field.

Starting communication with the inductive link and then changing to the RF link also provides a level of security in communicating with the AMD because the external device 406 must be near the patient to start the communication with the AMD. Unwanted RF communication with the AMD can be prevented because it is likely that the unwanted devices do not include an inductive link or are too far away to start a communication session.

There are different types of information that can be communicated between the AMD and the external device. Some information can be diagnostic information that is read from memory of the IMD. In an example, this type of information can be communicated by a medical device programmer or a monitoring-only device having an RF link, an inductive link, or both. Some information can be instructions sent to the AMD that can impact the patient (e.g., patient therapy parameters). In an example, this type of information can be communicated by a medical device programmer and not a monitoring-only device without an inductive link. The communications that may potentially impact the patient should have a higher level of security than communications that don't potentially impact the patient.

FIG. 5 is a flow diagram of an example of a method 500 of operating an AMD (e.g., the AMD of FIG. 3) to communicate with a second device (e.g., the external device 406 of FIG. 4). In the method 500, different instructions received by the AMD have different classes. For example, the instructions can be designated as class A instructions and class B instructions. A class A instruction may be an instruction that could potentially impact the patient by changing the operation of the AMD or that substantially impact the AMD in a meaningful way. In an example, class A instructions can be considered high-risk changes, including among others, programming changes that modify firmware, operating parameters, or therapy parameters of the AMD or instructions that result in substantive changes in AMD operation that can impact the usable lifespan of the AMD (e.g., lifespan impact greater than a threshold amount, etc.). A class B instruction may be an instruction that does not change the operation of the AMD (e.g., an instruction that only reads data from the AMD) or that does not impact the usable lifespan of the AMD above a threshold amount. For patient well-being, it may be desirable to send class A instructions with a higher level of security than class B instructions. Requiring class A instructions to be sent during communication sessions that were started with the inductive link provides a higher level of security in sending class A instructions.

In an example, requiring that programming of class A instructions is started using an inductive link can ensure that the programming is being done using a medical device programmer in a clinical setting. In certain examples, such clinical setting can require clinician involvement and proximity of medical device resources when making changes that can impact the physical safety or condition of the patient. Additionally, the medical device programmer can monitor patient condition pre- and post-programming change, such that additional programming changes can be triggered or suggested and changed by the medical device programmer based on a difference between the patient condition pre- and post-programming change.

In an example, pre- and post-programming changes can include, among other things, assessment of cardiac function, such as heart rate, rhythm, or cardiac performance, evaluation of patient condition, analysis of AMD performance metrics or impact on projected battery life of the AMD, etc. Requiring that high-risk changes be performed using a medical device programmer and conducting pre- and post-change evaluations can require oversight and minimize potential risks to patient safety and AMD operation, improving AMD efficacy.

At block 505, the AMD receives an instruction from the second device via the RF link of the AMD as part of a communication session. At block 510, the AMD determines the class of the instruction and whether the class requires that the instruction be sent during a communication session started with the inductive link.

At block 515, the AMD performs the instruction if the class of instruction requires that the communication session be started using the inductive link, and the communication session was indeed started using the inductive link. At block 520, the AMD rejects performing the instruction if the class of instruction requires that the communication session be started using the inductive link, and the communication session was started using the RF link instead of the inductive link.

If the instruction is rejected, the AMD may send a message to the second device indicating that the instruction was rejected. In certain examples, the AMD may send a message to the second device that the communication session should be restarted using the inductive link. In certain examples, the AMD may send no response to the second device when rejecting the instruction. When the AMD determines that the instruction is of a class that does not require that the instruction be sent during a communication session that was started using the inductive link (e.g., a class B instruction), the AMD performs the instruction when the communication session was started using either the inductive link or the RF link.

Security of communications with the AMD can be implemented using the external device. The external device can perform authentication of the user or a remote device before communications with the AMD are allowed. The requirement for using the inductive link for certain instructions adds a level of authentication to the AMD itself. This can be extended to other forms of authentication by the AMD. For instance, the firmware of the AMD may require authentication of the programming device before performing class A instructions.

The types of instruction that require using the inductive link (e.g., class A instructions) can be instructions that change the operation of the AMD when the instruction is performed. An example of a class A instruction is a programming instruction to program one or more operating parameters of the AMD. The AMD may include a therapy circuit 358 that provides an electrical therapy to the patient, and the programming instruction may change the value of one or more therapy parameters of the therapy the AMD provides to the patient. For instance, the AMD may be an IMD (e.g., IMD 102 in FIG. 2) that provides pacing therapy to the patient, and the AMD requires that an instruction that programs a pacing therapy parameter be sent during a communication session started with the inductive link. In another example, the AMD may provide high-energy defibrillation therapy to the patient, and the AMD requires that an instruction that programs a defibrillation parameter be sent during a communication session started with the inductive link. The therapy parameter may change the energy or timing of electrical therapy delivered to the patient, or the therapy parameter may be related to detection of a patient condition for delivering the therapy (e.g., detection of arrhythmia of the patient). In yet another example, a class A instruction can be an instruction for the AMD to perform a diagnostic test. These test instructions may cause the AMD to change operating parameters and monitor the results of the changing operating parameters by monitoring the output of one or more sensors of the IMD.

In a further example, a class A instruction can be an update instruction to update firmware in the AMD. Returning to FIG. 3, the AMD may include instructions 356 that are not changeable in the AMD. These instructions may be part of a safety core of instructions performable by the AMD. The AMD also includes firmware instructions 354 stored in a memory 352 of the AMD that may be separate from or integral to the control circuit 346 of the AMD. The control circuit 346 performs the firmware instructions to perform the functions described for the AMD. It may be desired to update the firmware instructions 354 of the AMD. The AMD may perform instructions to load new or updated firmware into the memory 352 during a communication session started with the inductive link.

Other examples of class A instructions can be instructions that don't directly impact the patient by changing the operation of the AMD but may impact battery life of the AMD. Communicating with a separate device uses battery energy of the AMD. Instructions that involve longer communication sessions may be limited to communications started with the inductive link to prevent unwanted RF communication sessions from negatively impacting the battery life of the AMD. For example, a class A instruction can be an upload instruction to upload information stored in the AMD to the second device. The AMD may require that the upload instruction be included in a communication session started with the inductive link when performing the instruction uploads an amount of information AMD greater than a predetermined threshold amount of information. This would prevent long instructions from being performed without an inductive link. In variations, a class A instruction can be any instruction that, when performed by the AMD, would result in a communication session lasting longer than a predetermined threshold amount of time.

Some examples of class B instructions not requiring starting the communication with the inductive link include read-only instructions, such as instructions to only read the programmed parameters of the AMD, or to upload results of diagnostic tests performed by the AMD.

In some examples, the AMD may also require authentication of the second device to perform class A instructions. For instance, the AMD may require that the second device prove that it has the role of medical device programmer to perform a class A instruction. To authenticate the second device, the second device may send a predetermined authentication key to the AMD during the communication session. The AMD uses the authentication key to verify the role of the second device and performs the class A instruction when the key verifies the role of the second device and the communication session is started with the inductive link. In some examples, an instruction to write authentication key or keys in memory of the AMD for the AMD to use in authenticating other devices may be a class A instruction, and the key or keys are programmed during a communication session that was started with the inductive link.

Communication sessions between an AMD and an external device can become interrupted, e.g., due to noise, movement of the patient, etc. If the communication session includes class A instructions, the communication session may need to be restarted using the inductive link to maintain the higher level of security. In some examples, the communication session can be maintained without having to be restarted if an inductive signal is received by the AMD within a predetermined amount of time. For instance, the control circuit 346 of the AMD in FIG. 3 can include a timeout timer 348. During a communication session, the second device may periodically send a special character or characters via the RF link during a communication session to maintain alignment of data being transferred on the RF link or to maintain communication while the AMD performs a class A instruction. The AMD may detect interruption of the RF communication if the AMD does not receive a special character within a specified amount of time after the last special character. The AMD may interrupt performance of the instruction in response to detecting the interruption of the RF communication signal. The AMD may require a near-field signal to be received on the inductive link before resuming one or both of the communication session and the performance of the instruction. If the timeout timer expires before the near-field signal is received, the AMD may require that a new communication session be started.

As described previously herein, the AMD performs instructions of a specific class if the instructions are sent during a communication session started with the inductive link, and rejects performing the class of instructions if the communication session was started using the RF link.

According to some examples, this requirement of the inductive link can be overridden by the second device. Certain devices may be allowed to perform one or more of the class A instructions using the RF link without starting the communication with the inductive link. For example, it may be desired to use a remote server to update the firmware of multiple AMDs. Certain devices may be given roles having Remote Operations privileges or Developer privileges. These role privileges may allow the inductive link requirement to be overridden. In another example, a physician may be allowed to perform class A instructions remotely after the physician is authenticated. The authentication may be performed by a remote server, an external device located with the patient, or by the AMD.

To accept a class A instruction from a second device without first receiving a near-field signal from the inductive link, the AMD may authenticate the role and privileges of the second device. When the AMD authenticates that the second device has the appropriate role (e.g., using an authentication key sent by the second device), the AMD may override the rejecting the instruction according to the role of the second device and perform the instruction when the communication session is started using the RF link. In certain examples, the AMD may authenticate a medical device programmer when the medical device programmer sends the appropriate authentication key for a role of medical device programmer. This is another way to authenticate the clinical presence of the programming device without using an inductive link.

Different programming profiles can be assigned to different roles. The profiles can be predetermined, and each profile may include a specific subset of parameters that are made programmable. The role of a user or device defines the profiles that are made available for remote programming. The profiles may address scenarios that arise that use specific programming. For example, an “Ensure Pacing” profile may allow maximum pacing outputs to be set by a user or device having the proper role or privileges. In some examples, the profiles include predetermined parameters that are set when the profile is selected by the external device. For example, an “Electrocautery” profile or “MRI Mode” profile automatically sets specific operating parameters to support surgery or diagnostic needs. A “Disable Therapy” profile may be used for emergency situations or hospice situations. An “Onset of AF” profile may enable atrial fibrillation diagnostics to support diagnostics of a new AF occurrence in AF patients.

The several examples of AMDs described herein provide a higher level of security when communicating with the AMDs using RF communications. Certain classes of instruction provide convenience to the patient and physician by being RF only, and some classes of instructions can only be performed when some level of authentication is performed by the AMD, such as if an inductive communication link is used for example.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The term “transmission medium” includes any intangible medium that is capable of storing, encoding, or carrying instructions for execution by a machine, and includes digital or analog communications signals or other intangible medium to facilitate communication of such software. A transmission medium is a machine-readable medium.

The above detailed description is intended to be illustrative, and not restrictive. The scope of the disclosure should, therefore, be determined with references to the appended claims, along with the full scope of equivalents to which such claims are entitled.

What is claimed is:

1. An ambulatory medical device (AMD), the device comprising:
an inductive link including a coil antenna configured to receive a near field communication signal using mutual inductance;
a radio frequency (RF) link including an RF antenna configured to receive an RF communication signal during a communication session;
a control circuit configured to:
decode an instruction received in the RF communication signal, wherein a class of the instruction requires the communication session to be started using the inductive link when performing the instruction;
perform the instruction when the communication session is started using the inductive link; and
reject the instruction when the communication session is started using the RF link.

2. The AMD of claim 1, wherein the control circuit is configured to:
decode another instruction received in another RF communication signal during another communication session, wherein a class of the other instruction does not require the communication session to be started using the inductive link when performing the instruction; and
perform the other instruction when the communication session is started using either the RF link or the inductive link.

3. The AMD of claim 1, wherein the control circuit is configured to:
authenticate a role of a second device sending the RF communication signal; and
override the rejecting the instruction according to the role of the second device and perform the instruction when the communication session is started using the RF link.

4. The AMD of claim 1, wherein the control circuit is configured to:
determine that the instruction received in the RF communication signal is a programming instruction to program one or more operating parameters of the AMD;
perform the programming instruction when the communication session is started using the inductive link; and
reject the programming instruction when the communication session is started using the RF link.

5. The AMD of claim 1, including:
a therapy circuit operatively coupled to the control circuit and configured to provide an electrical therapy; and
wherein the control circuit is configured to:
determine that the instruction received in the RF communication signal is a programming instruction to program a value of a therapy parameter of the electrical therapy provided by the AMD;
perform the programming instruction when the communication session is started using the inductive link; and
reject the programming instruction when the communication session is started using the RF link.

6. The AMD of claim 1, wherein the control circuit is configured to:
determine that the instruction received in the RF communication signal is an upload instruction to upload an amount of information stored in the AMD greater than a predetermined threshold amount of information;
perform the upload instruction when the communication session is started using the inductive link; and
reject the upload instruction when the communication session is started using the RF link.

7. The AMD of claim 1, wherein the control circuit is configured to:
determine that the instruction received in the RF communication signal is an instruction that when performed results in a communication session lasting longer than a predetermined threshold amount of time;
perform the instruction when the communication session is started using the inductive link; and
reject the instruction when the communication session is started using the RF link.

8. The AMD of claim 1, wherein the control circuit is configured to:
determine that the instruction received in the RF communication signal is an update instruction to update firmware in the AMD;
perform the update instruction when the communication session is started using the inductive link; and
reject the update instruction when the communication session is started using the RF link.

9. The AMD of claim 1, wherein the control circuit includes a timeout timer and is configured to:
begin performing the instruction when the communication session is started using the inductive link;
detect interruption of an RF communication signal on the RF link; and
resume performing the instruction when another near field communication signal is received on the inductive link before the timeout timer expires.

10. The AMD of claim 1, wherein the control circuit is configured to:
authenticate a role of a second device sending the RF communication signal; and
override the rejecting the instruction when the role of the second device is authenticated, and the instruction programs a parameter included in a profile of parameters enabled for programming according to the role of the second device.

11. The AMD of claim 1, wherein the control circuit is configured to:
authenticate a role of a second device sending the RF communication signal; and
perform the instruction when the communication session is started using the RF link and the role of the second device is authenticated.

12. The AMD of claim 1, wherein the control circuit is configured to perform the instruction when the communication session is started using the RF link and the instruction enables a predetermined profile of operating parameters of the AMD.

13. A method of operating an ambulatory medical device (AMD) to communicate with an external device, the method comprising:
receiving an instruction via a radio frequency (RF) communication link of the AMD as part of a communication session with a second device;
determining, by the AMD, that a class of the received instruction requires the communication session to be started using an inductive link of the AMD when performing the instruction;
performing the instruction when the communication session is started using the inductive link; and
rejecting the instruction when the communication session is started using the RF link.

14. The method of claim 13, including:
receiving a different instruction via the RF link from the second device, wherein a class of the different instruction does not require the communication session to be started using the inductive link when performing the instruction; and
performing, by the AMD, the different instruction when the communication session is started using either the RF link or the inductive link.

15. The method of claim 13, including:
authenticating a role of the second device; and
overriding the rejecting the instruction according to the role of the second device and performing the instruction when the communication session is started using the RF link.

16. The method of claim 13,
wherein the determining that a class of the received instruction requires the communication session to be started using the inductive link includes determining that the instruction received via the RF link is a programming instruction to program one or more operating parameters of the AMD and requiring the communication session to be started using the inductive link to perform the programming instruction.

17. The method of claim 13,
wherein the determining that a class of the received instruction requires the communication session to be started using the inductive link includes determining that the instruction received via the RF link is a programming instruction to program a value of a therapy parameter of an electrical therapy provided by the AMD and requiring the communication session to be started using the inductive link to perform the programming instruction.

18. The method of claim 13,
wherein the determining that a class of the received instruction requires the communication session to be started using the inductive link includes determining that the instruction received via the RF link is an upload instruction to upload an amount of information stored in the AMD greater than a predetermined threshold amount of information requiring the communication session to be started using the inductive link to perform the upload instruction.

19. An external medical device comprising:
an inductive link including a coil antenna configured to transmit a near field communication signal to an ambulatory medical device (AMD) using mutual inductance;
a radio frequency (RF) link including an RF antenna configured to transmit an RF communication signal with the AMD;
a control circuit operatively coupled to the inductive link and the RF link and configured to:
begin a communication session with the AMD using the inductive link when sending a first class of instruction to the AMD;
begin the communication session with the AMD using the RF link when sending a second class of instruction to the AMD; and
send authentication information to the AMD during the communication session for authentication of the external medical device.

20. The external medical device of claim 19, wherein the control circuit is configured to:
begin the communication session with the AMD using the near field communication signal when sending a programming instruction to program one or more operating parameters of the AMD.
